(19) United States
(12) Patent Application Publication
(10) Pub. No.: US 2018/0275119 A1
(43) Pub. Date: Sep. 27, 2018

(54) METHODS AND DEVICES FOR DETECTING PROTEIN IN SALIVA SAMPLES

(71) Applicant: SLIVE, INC., Palo Alto, CA (US)

(72) Inventors: Erez Podoly, Menlo Park, CA (US); Anjay Saklecha, San Jose, CA (US)

(21) Appl. No.: 15/933,311

(22) Filed: Mar. 22, 2018

Related U.S. Application Data
(60) Provisional application No. 62/475,209, filed on Mar. 22, 2017.

(51) Int. Cl. G01N 33/53; C07K 14/705; G01N 33/68; A61B 10/00

(52) U.S. Cl. CPC G01N 33/5306; C07K 14/705; A61B 2505/01; A61B 10/0051; G01N 2333/47; G01N 33/6896

(57) ABSTRACT

Methods and devices for detecting proteins in saliva are disclosed. The methods comprise obtaining a saliva sample from a subject suspected of having a traumatic brain injury (TBI). The sample is probed for a biomarker associated with TBI. The subject's biomarker profile is then compared to a reference sample or to a threshold value. The disclosed devices are able to detect in realtime the presence of biomarker proteins associated with TBI.

### CROSS REFERENCE TO RELATED APPLICATION

This application claims priority to U.S. Provisional Application Ser. No. 62/475,209, filed Mar. 22, 2017, titled METHODS FOR DETECTING PROTEINS IN SALIVA, the disclosure of which is incorporated herein in its entirety.

### FIELD OF THE INVENTION

Disclosed herein are methods and devices for detecting proteins in saliva samples. More particularly, the methods and devices described herein relate to detecting proteins associated with brain injury in saliva samples.

### BACKGROUND OF THE INVENTION

Traumatic brain injuries are increasingly recognized as a serious health issue in the United States with the Centers for Disease Control estimating that 2.5 million people suffered a traumatic brain injury (TBI) in 2010. TBI is caused by an external force applied to the head that disrupts the normal functions of the brain. In mild TBI cases, the subject may have a brief altered state of consciousness or changes in mental functioning while severe TBI is often accompanied by extended periods of unconsciousness, emotional changes, impaired cognitive function, and/or impaired motor function.

Concussions are a mild form of TBI and have become an increasing cause of concern due to recent findings that link concussions with chronic traumatic encephalitis (CTE). Due to the concussive impacts caused by implements of warfare and sustained by soldiers, the U.S. military has a strong felt need for methods and devices for detecting concussions and TBI. Participants in contact sports such as football also have justified concerns regarding TBI, especially in light of the growing number of former college and professional football players who sustained multiple concussions during their playing careers and were diagnosed with advanced CTE postmortem. The cognitive and motor issues associated with concussions and TBI is a major concern for players, parents, youth leagues, the NCAA, and the NFL. Currently, people suspected of having sustained some form of TBI are evaluated “in the field” by physicians who assess the person's symptoms (i.e., difficulty thinking, nausea, etc.). These qualitative assessments are vulnerable to human error and even societal pressures such as a soldier wanting to rejoin his fellow combatants or a player wanting to rejoin his teammates following an injury.

Recent research has shown that some proteins can be detected in blood samples, and are indicative of TBI, after an injury. For example, WO 2014/004424, the disclosures of which are hereby incorporated herein by reference in their entirety, teaches that brain endothelial tight junction complexes are comprised of proteins such as occludin, claudin (claudin 3, 5, 12), zonula occludens protein (ZO-1, 2, 3), and junctional adhesion molecules (JAM-A, B, C). WO 2014/004424 also teaches patients can be screened for brain injury by testing for the presence of these proteins in blood, blood plasma, blood serum, cerebrospinal fluid or urine samples from a subject. It is invasive or inconvenient to obtain each of these samples from a person potentially suffering from a brain injury.

Thus, there is a need for noninvasive techniques and devices for detecting the presence or absence of proteins associated with brain injury in a subject. The disclosed methods and devices are directed to these and other important needs.

### SUMMARY OF THE INVENTION

Disclosed herein are methods for detecting proteins associated with brain injury in a subject comprising obtaining a saliva sample originating from the subject and detecting the presence or absence of occludin in the sample.

Methods are also disclosed for detecting brain injury in a human subject comprising obtaining a saliva sample originating from the human subject; contacting the sample with an antibody, salimer, or other molecule with high affinity for at least one protein associated with brain injury, wherein the antibody, salimer, or other molecule are not naturally occurring in humans; and diagnosing the human subject with a brain injury when the presence of the at least one protein associated with brain injury is detected in the saliva sample.

Also disclosed herein are devices for detecting brain injury in a subject comprising a mouthpiece having a plurality of reaction chambers, each reaction chamber being in fluid communication with the oral cavity via an inlet and comprising at least one reactant capable of generating a signal in the presence of at least one protein associated with brain injury.

### BRIEF DESCRIPTION OF THE DRAWINGS

The summary, as well as the following detailed description, is further understood when read in conjunction with the appended drawings. For the purpose of illustrating the disclosed methods and devices, there are shown in the drawings exemplary embodiments of the methods and devices; however, the methods and devices are not limited to the specific embodiments disclosed. In the drawings:

FIG. 1 depicts the level of occludin observed in patient sets comprising individuals with no TBI, moderate TBI, and severe TBI.

### DETAILED DESCRIPTION OF ILLUSTRATIVE EMBODIMENTS

The disclosed methods and devices may be understood more readily by reference to the following detailed description taken in connection with the accompanying figures, which form a part of this disclosure. It is to be understood that the disclosed methods and devices are not limited to the specific methods and devices described and/or shown herein, and that the terminology used herein is for the purpose of describing particular embodiments by way of example only and is not intended to be limiting of the claimed methods and devices.

Unless specifically stated otherwise, any description as to a possible mechanism or mode of action or reason for improvement is meant to be illustrative only, and the disclosed methods and devices are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods and devices which are, for clarity, described herein in the context of separate embodiments, may also be provided in combination in a single embodiment. Conversely, various features of the disclosed methods and devices that are, for brevity, described in the context of a single embodiment, may also be provided separately or in any subcombination.

As used herein, the singular forms “a,” “an,” and “the” include the plural.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

As used herein, “brain injury,” “traumatic brain injury,” and TBI are used interchangeably.

A “salimer” is a single-stranded oligonucleotide that comprises a deoxyribonucleotide, a ribonucleotide, a peptide nucleotide, a morpholino, a locked nucleotide, a glycol nucleotide, a threose nucleotide, nucleotides phosphoramidite, any synthetic nucleotides, or any isoforms, combinations, or derivatives thereof.

The term “subject” as used herein is intended to mean any animal, in particular, mammals. Although detection of proteins associated with TBI in humans is exemplified herein, any type of mammal can be treated using the disclosed methods. Thus, the methods are applicable to human and nonhuman animals, although preferably used with mammals, and most preferably with humans. “Subject” and “patient” are used interchangeably herein.

As used herein, “percent identity” and like terms are used to describe the sequence relationships between two or more nucleic acids, polynucleotides, proteins, or polypeptides, and is understood in the context of and in conjunction with the terms including: (a) reference sequence, (b) comparison window, (c) sequence identity and (d) percentage of sequence identity.
(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence.
(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Those of skill in the art understand that to avoid a misleadingly high similarity to a reference sequence due to inclusion of gaps in the polynucleotide sequence a gap penalty is typically introduced and is subtracted from the number of matches.
(c) Methods of alignment of sequences for comparison are well known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which may be used for database similarity searches includes: BLASTN for nucleotide query sequences against nucleotide database sequences; BLASTX for nucleotide query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and may be used with the present disclosure.
(d) “Percent identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

Various terms relating to aspects of the description are used throughout the specification and claims. Such terms are to be given their ordinary meaning in the art unless otherwise indicated. Other specifically defined terms are to be construed in a manner consistent with the definitions provided herein.

Concussions are a major concern in modern life due to impacts suffered by humans during the course of perceived normal activities such as participating in contact sports, driving, hiking, walking, or any other of a multitude of endeavors that have a risk of sustaining a head injury. Concussions and other forms of TBI are also often-incurred injuries on the modern battlefield. Accurate yet non-invasive tests for concussions and TBI are necessary for detecting and monitoring proteins associated with TBI in a subject at risk of, suspected of having, or confirmed to have a concussion or other TBI.

The present disclosures provide for methods and devices that allow the convenient, non-invasive detection of brain injuries. By requiring only a saliva sample, the present disclosures avoid more intrusive techniques such as blood draws or less convenient protocols that involve collection of urine samples. The premise of the disclosures is that once a TBI is suspected to have occurred, specific proteins are released and detectable in saliva. Detecting these proteins associated with TBI allows for the effective and efficient detection of brain injury in a subject.

Thus, one embodiment of the present disclosures provides a method for detecting a protein associated with brain injury in a subject comprising obtaining a saliva sample originating from the subject; and detecting the presence or absence of the protein associated with brain injury in the sample. Obtaining a saliva sample from the subject can be accomplished by directly taking a saliva sample from the subject or by obtaining the sample from a third party in possession of the subject's saliva sample. Collecting a saliva sample can be accomplished in any manner known in the art so long as the sample originates from the subject and at least a sufficient amount of saliva is collected. A sufficient amount of saliva is the minimum amount required to perform the detection assay or assays.

Detecting the presence or absence of a protein associated with brain injury, such as occludin in the sample can be accomplished using known techniques such as, but not limited to, flow cytometry, enzyme linked immunosorbent assay (ELISA), and high performance electrophoresis (HPLC). In other embodiments of the present disclosures, an immunoassay is used for detecting the presence or absence of a protein associated with brain injury. In some aspects, the protein associated with brain injury is occludin. In some aspects of these embodiments, the immunoassay employs an antibody that is not naturally occurring in humans. The antibody may be naturally occurring in another species distinct from humans or the antibody may be artificial or recombinant. Such artificial or recombinant antibodies may be manufactured using techniques known in the art. In other embodiments, a molecule with high affinity for a protein associated with brain injury and that is not naturally occurring in humans is employed to detect the presence or absence of a protein associated with brain injury. In some aspects, the molecule with affinity for a protein associated with brain injury is an oligonucleotide. In some aspects the oligonucleotides may comprise at least one non-naturally occurring or synthetic nucleotide.

The methods described herein may further comprise simultaneously detecting and quantifying a protein associated with brain injury in a reference sample. A “reference sample” as used herein refers to any sample which can be used to make qualitative and/or quantitative comparisons. For example, a reference sample may include a purified sample of a known concentration of a protein associated with brain injury (e.g., occludin) such that a positive result for the subject's sample can be compared to the reference sample and the concentration of a protein associated with brain injury in the reference sample can be estimated.

In some of embodiments of the methods provided herein, obtaining the saliva sample and detecting the presence or absence of the protein of interest are performed in the subject's mouth. In one aspect, the method will employ an insertable device comprising the reagents necessary to detect proteins associated with brain injury, such as occludin. One non-limiting example of an insertable device is a tongue depressor equipped to house the reagents necessary to detect the proteins of interest. The tongue depressor may be disposable and suitable for single or multiple use. For some aspects of the multiple use tongue depressors, the depressor will house multiple reaction chambers. Each reaction chamber houses the reagents necessary for detecting the proteins associated with brain injury when exposed to the patient's saliva. The reaction chambers on the tongue depressor may be exposed to the saliva sample simultaneously to give multiple parallel results. Alternatively, different reaction chambers may be exposed to the saliva sample at different time points after, or before and after, the injury in order to monitor the levels of proteins over time. Furthermore, the reaction chambers may be exposed to saliva samples over time to surveil for proteins associated with TBI.

In other aspects, the method of detecting brain injuries will employ a wearable device such as a mouth guard. Because mouth guards are often worn during contact sports such as football and basketball, the mouth guards can be used for real-time detection and monitoring of brain injury at the time of injury. Presumably, an individual wearing a mouth guard capable of detecting occludin or other proteins would have low levels of the protein(s) prior to participating in a contact sport or other activity with a risk of injury causing increased levels of occludin or other proteins in a saliva sample. While wearing the mouth guard, one would be able to monitor occludin or other proteins levels in the individual in real time.

In some aspects, the patient's saliva sample is processed such that the proteins of interest in the sample are able to be identified. One example is a Western blot in which the proteins in the sample are resolved on a polyacrylamide gel, transferred to nitrocellulose membrane or other substrate which is then exposed to labeled antibodies specific for a protein of interest like occludin. The labeled antibody emits a detectable signal that when exposed to film allows for the visual confirmation of the presence of the protein in the sample. Accordingly, in some aspects of the disclosures, detecting the presence of occludin in the sample results in an optical signal. In some aspects, the optical signal is a fluorescent, luminescent, phosphorescent, or other visual signal.

In other embodiments of the present disclosures, an electrical signal is generated upon detection of occludin or another protein of interest. Therefore, one aspect of the disclosures is detecting the presence of a protein associated with brain injury in the sample results in an electrical signal. In some aspects, the electrical signal is generated by the interaction of a labeled reagent, such as a metal labeled antibody, with an electrode. In other aspects, a molecule or compound having a higher affinity for a protein associated with brain injury, or another protein or molecule of interest, than to a second molecule or compound disposed at the surface of an electrode can generate an electrical signal in the presence of a protein associated with brain injury, such as occludin. For example, prior to exposure to the saliva sample, the molecule or compound having affinity for a protein associated with brain injury will interact with the molecule or compound tethered to the electrode. Upon exposure to a protein associated with brain injury, the molecule or compound having a higher affinity for a protein associated with brain injury will disassociate from the tethered molecule or compound, and this will generate an electrical signal indicating the presence of a protein associated with brain injury in the sample. Because there will be a plurality of molecules or compounds tethered to the electrode and a plurality of molecules or compounds having a higher affinity for a protein associated with brain injury, the signal is proportional to the amount of a protein associated with brain injury detected in the sample.

Some embodiments of the present disclosures in which an electrical signal is generated further comprise transmitting the electrical signal to a receiver. By transmitting the signal to a receiver, the data from the signal can be analyzed to determine not only qualitative properties such as “present” or “absent,” but can also be analyzed to determine quantitative properties such as strength of the signal. Estimates of the amount of a protein associated with brain injury in the sample can be determined when the signal is proportional or related to the amount of a protein associated with brain injury in the sample.

The methods described above can further comprise performing the steps in an iterative fashion over a period of time. This enables one to determine the amount of a protein associated with brain injury in the saliva of a patient over a period of time, allowing caregivers in some circumstances to estimate the severity of the injury, the time elapsed since the injury, and the patient's response to therapy. Similarly, the methods described above can further comprise quantifying the amount of a protein associated with brain injury in the sample obtained from the patient.

Another embodiment of the present disclosures provides a method of detecting brain injury in a human subject comprising obtaining a saliva sample originating from the human subject; contacting the sample with an antibody, salimer, or other molecule with high affinity for a protein associated with brain injury, wherein the antibody, salimer, or other molecule are not naturally occurring in humans; and diagnosing the human subject with a brain injury when the protein associated with brain injury is detected in the saliva sample.

Some embodiments of the method further comprise detecting a signal after contacting the sample with an antibody, salimer, or other molecule with high affinity for the protein associated with brain injury. In some aspects the signal is an optical, electrical, thermal, gravimetrical, pH, or radioactive emission signal.

When detecting a protein associated with brain injury, the signal indicates the presence or absence of the protein associated with brain injury. The intensity of the signal detected is proportional to the amount of the protein associated with brain injury present in the sample in some embodiments. Thus, based on the intensity of the signal detected, one skilled in the art would be able to determine the amount of the protein associated with brain injury in the sample. The amount of the protein associated with brain injury in the sample may be determinative of a brain injury. Alternatively, the amount of the protein associated with brain injury in the sample may allow one skilled in the art to extrapolate when the injury occurred, the severity of the initial injury, or if the injury is responding to therapy.

Reference samples comprising known amounts of occludin (or another protein of interest) may be used to gauge the level of occludin in a test sample. In some embodiments, the methods described herein further comprise detecting the presence of the protein associated with brain injury in a reference sample and comparing the detected amounts of the protein associated with brain injury in the reference sample and the saliva sample from the human subject. The reference sample may comprise an amount of the protein of interest that is indicative of brain injury so that if the test sample contains an equivalent or greater amount than the reference sample, a diagnosis of brain injury is reasonable. Similarly, parallel reference samples having different amounts of the protein of interest can be used to generate signals proportional to the different concentrations of protein. One skilled in the art can then estimate the concentration of the protein of interest in the subject's sample based on similarities and differences between the subject sample and the reference samples.

Concentration of the protein of interest in the subject sample can be measured using any number of well-known techniques in the art. Many techniques, such as spectrophotometry, will require equipment usually available in only laboratory settings. Other techniques, such as visual comparison of fluorescent signals, can be performed more cheaply and in non-laboratory environments.

Some embodiments of the methods for diagnosing brain injury as described herein may further comprise obtaining at least one additional saliva sample and contacting the additional sample with the antibody, salimer, or other molecule with high affinity for a protein associated with brain injury protein. In some aspects, obtaining the at least one additional sample happens simultaneously with the original sample. This additional sample can be used as a control against false positive and/or false negative results of the initial sample. In other aspects, obtaining the at least one additional sample occurs at some time after the initial sample is collected and analyzed. This will allow one to monitor, among other things, the progression of the injury, response to therapy, and even estimate based on the rate of change in the concentration of the protein of interest how severe the initial injury was. Some embodiments further provide for quantitating the amount of the protein associated with brain injury detected for each additional saliva sample obtained and/or comparing the amount of the protein associated with brain injury detected at each assayed time and determining the effect of time on the concentration of the protein associated with brain injury in the samples.

Other embodiments of the present disclosures provide for devices for detecting brain injury in a subject comprising a mouthpiece having a plurality of reaction chambers, each reaction chamber being in fluid communication with the oral cavity via an inlet and comprising at least one reactant capable of generating a signal in the presence of a protein associated with brain injury. In some aspects, the mouthpiece can be worn as a mouth guard or even incorporated into orthodontics to allow for monitoring of proteins associated with brain injury. In some aspects and in the presence of at least one protein associated with brain injury, the device will be capable of emitting a signal alerting the detection of the at least one protein.

The device, in some embodiments, comprise multiple reactants that can detect proteins of interest. For example, in some embodiments some reactants may be involved in the detection of a protein associated with brain injury, such as occludin, while other reagents are involved in the detection of other proteins involved in tight junctions. Similarly, in some embodiments of the disclosures, some reagents comprising the device augment the signal generated by other reagents or convert the signal from some reagents to a different signal. For example, a change in pH in the reaction chamber may be a signal that a protein of interest has been detected. A change in pH can be quantified using a pH meter, but in some aspects, another reagent, sensitive to changes in pH, would emit an optical signal in response to the change of pH.

In some embodiments of the present disclosures, the at least one reactant is a salimer having a higher affinity for a protein associated with brain injury than for a primer with which the salimer can hybridize. In some aspects, the reaction chamber further comprises the primer with which the salimer can hybridize. When the reaction chamber comprises both the primer and the salimer, a competition reaction ensues between the primer and any protein of interest for which the salimer has an affinity for binding to the salimer. In some aspects, binding of the salimer to the protein will generate a signal. For example, in some aspects, the primer is immobilized to the surface of an electrode, the primer and the salimer are hybridized prior to exposure to the protein associated with brain injury, and dehybridization of the primer and the salimer causes the electrode to generate an electrical signal.

The primer and the salimer will at least partially hybridize because they have at least some sequence homology. In some aspects the salimer and the primer have about 100%, 90%, 80%, 70%, 60%, 50%, 40%, 30%, 20%, 10%, and even 5% sequence homology. In some aspects, the salimer and the primer have about 95% sequence homology. In some aspects, the salimer and the primer have about 75% homology. In still other aspects, the salimer has about 50% sequence homology. In some aspects, the primer and the salimer have at least 50% sequence homology.

In some embodiments of the present disclosures, the primer forms a secondary structure when it is single-stranded. Such a secondary structure is not possible if the primer is hybridized to the salimer. Upon disassociation of the salimer-primer hybrid, the single-stranded primer will assume its secondary structure, and this property can be utilized to augment the signal generated when the salimer disassociates from the primer. In some embodiments, the primer will have a label disposed at the opposite end of the primer from the end that is disposed on the surface of the electrode. As the primer assumes the secondary conformation, the label is positioned closer to the electrode.

In some aspects of the present disclosures, the devices are designed so that they can be worn or disposed of in the mouth of the subject, and because they are worn at the time of injury, confirmation or detection of the brain injury is nearly simultaneous with the injury itself. In some injuries, the levels of proteins associated with brain injury may only accumulate after a period of time post-injury, and in some aspects the device will monitor the accumulation so that proper precautions and medical treatment are provided to improve the health status of the injured subject.

Because the devices can be worn continuously, as in the cases of orthodontics or mouth guards, saliva must be removed from the device so that newly collected saliva can enter the reaction chambers. For this reason, some embodiments of the present disclosures further comprise an outlet so that saliva is moved nearly continuously through the device when inserted into the subject's mouth.

The protein associated with brain injury is occludin in some embodiments of the present disclosure. In other embodiments the protein associated with brain injury may be claudin-3, 5, or 12; ZO-1, 2, or 3; and/or JAM-A, B, or C. Thus, in one aspect the protein associated with brain injury is claudin-3. In one aspect, the protein associated with brain injury is claudin-5. In one aspect, the protein associated with brain injury is claudin-12. In one aspect, the protein associated with brain injury is ZO-1. In one aspect, the protein associated with brain injury is ZO-2. In one aspect, the protein associated with brain injury is ZO-3. In one aspect, the protein associated with brain injury is JAM-A. In one aspect, the protein associated with brain injury is JAM-B. In one aspect, the protein associated with brain injury is JAM-C.

### EXAMPLES

The following examples are provided to further describe some of the embodiments disclosed herein. The examples are intended to illustrate, not to limit, the disclosed embodiments.

### Example 1: Detection of Occludin in Severe Brain Injuries

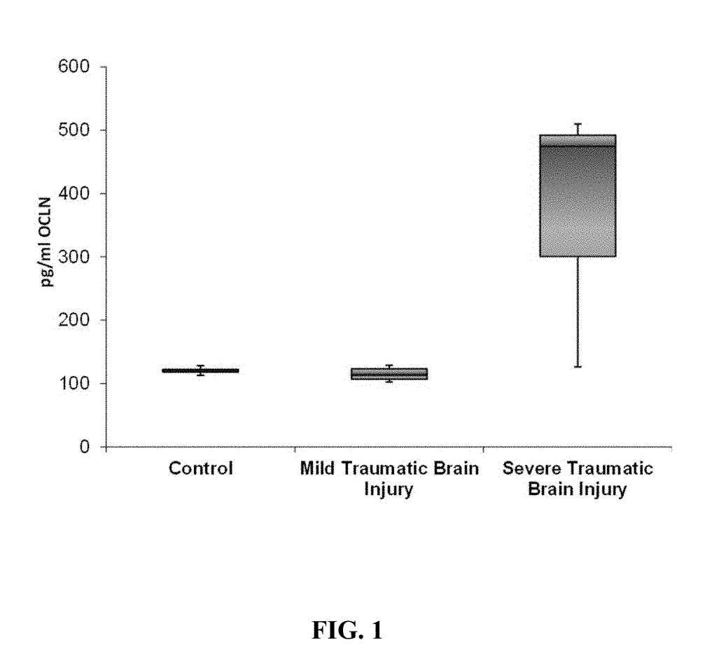

To determine if occludin could be detected in saliva, samples were obtained from subjects with no brain injury (n=10), subjects with mild brain injury (n=7), and subjects with moderate (n=1) and severe brain injury (n=2). The moderate and severely injured subjects arrived at an emergency room shortly after injury was sustained and were assessed with CAT scans to confirm brain injury and are grouped together in FIG. 1 as “Severe Traumatic Brain Injury.”

Some of the mild traumatic brain injury subjects did not seek immediate medical help but were confirmed by medical personnel to have sustained a mild brain injury based on their symptomology. The time lag between injury and seeking medical help may contribute to the lower levels of occludin detected in the “Mild Traumatic Brain Injury” subjects.

ELISA analysis of the saliva samples were performed using commercially available kits for detecting occludin. FIG. 1 depicts the results of the assays for the three groups of subjects. Subjects diagnosed with mild traumatic brain injury showed little if any additional occludin in their saliva samples compared to the control subjects (approx. 100 pg/mL). Subject with severe brain injury, however showed an appreciable increase in occludin concentration in their saliva samples (approx. 300-500 pg/mL).

Because those with mild traumatic brain injury did not seek immediate medical help, the analysis of their saliva samples also occurred at a later time after sustaining injury than did the analysis for the severely injured subjects. These results support the proposition that analyzing saliva for indications of brain injury is an effective and time sensitive mechanism for assessing brain injury in humans.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed:

1. A method for detecting a protein associated with brain injury in a subject comprising:
a) obtaining a saliva sample originating from the subject; and
b) detecting the presence or absence of the protein associated with brain injury in the sample.

2. The method according to claim 1, wherein an immunoassay is used for detecting the presence or absence of the protein associated with brain injury.

3. The method of claim 2, wherein an antibody or molecule with high affinity for the protein associated with brain injury is employed to detect the presence or absence of occludin.

4. The method of claim 3, wherein the molecule with high affinity for the protein associated with brain injury is an oligonucleotide.

5. The method of claim 1 further comprising simultaneously detecting and quantifying the protein associated with brain injury in a reference sample.

6. The method of the claim 1, wherein obtaining the saliva sample and detecting the presence or absence of the protein associated with brain injury are performed in the subject's mouth.

7. The method of claim 1, wherein detecting the presence of the protein associated with brain injury in the sample results in a signal.

8. The method of claim 7, wherein the signal is a fluorescent, luminescent, phosphorescent, or other visual signal.

9. The method of claim 1, wherein detecting the presence of the protein associated with brain injury in the sample results in an electrical signal.

10. The method of claim 1 further comprising quantifying the amount of protein associated with brain injury in the sample.

11. A method of detecting brain injury in a human subject comprising:
a) obtaining a saliva sample originating from the human subject;
b) contacting the sample with an antibody, salimer, or other molecule with high affinity for a protein associated with brain injury, wherein the antibody, salimer, or other molecule are not naturally occurring in humans; and
c) diagnosing the human subject with a brain injury when the presence of the protein associated with brain injury is detected in the saliva sample.

12. The method of claim 11 further comprising detecting a signal after contacting the sample with an antibody, salimer, or other molecule with high affinity for the protein associated with brain injury.

13. The method of claim 11, wherein the signal is an optical electrical, thermal, gravimetrical, or pH signal, or radioactive emission signal.

14. The method of claim 13, wherein the signal indicates the presence of the protein associated with brain injury.

15. The method of claim 11 further comprising detecting the presence of the protein associated with brain injury in a reference sample and comparing the detected amounts of the protein associated with brain injury in the reference sample and the saliva sample from the human subject.

16. The method of claim 11 further comprising obtaining at least one additional saliva sample at a later time point and contacting the additional sample with the antibody, salimer, or other molecule with high affinity for the protein associated with brain injury.

17. A device for detecting brain injury in a subject comprising:
a mouthpiece having a plurality of reaction chambers, each reaction chamber being in fluid communication with the oral cavity via an inlet and comprising at least one reactant capable of generating a signal in the presence of a protein associated with brain injury.

18. The device of claim 17, wherein the at least one reactant is a salimer having a higher affinity for the protein associated with brain injury than for a primer with which the salimer can hybridize.

19. The device of claim 17, wherein each reaction chamber further comprises the primer with which the salimer can hybridize.

20. The device of claim 17, wherein the primer is immobilized to the surface of an electrode, the primer and the salimer are hybridized prior to exposure to the saliva sample, and dehybridization of the primer and the salimer causes the electrode to generate an electrical signal.
